(19) United States
(12) Patent Application Publication
(10) Pub. No.: US 2021/0008506 A1
(43) Pub. Date: Jan. 14, 2021

(54) HYBRID MATERIALS FOR ORGANIC SOLVENT NANOFILTRATION AND PERVAPORATION MEMBRANES

(71) Applicant: King Abdullah University of Science and Technology, Thuwal (SA)

(72) Inventors: Tiara PUSPASARI, Thuwal (SA); Tina CHAKRABARTY, Thuwal (SA); Klaus-Viktor PEINEMANN, Thuwal (SA)

(21) Appl. No.: 16/977,228

(22) PCT Filed: Mar. 3, 2019
(86) PCT No.: PCT/IB2019/051728

§ 371 Date: Sep. 1, 2020

Related U.S. Application Data
(60) Provisional application No. 62/638,500, filed on Mar. 5, 2018.
(60) Provisional application No. 62/749,873, filed on Oct. 24, 2018.

(51) Int. Cl. B01D 71/70; C08L 1/02; C08L 83/04; C08J 5/18; C08J 9/00; B01D 61/02; B01D 61/36; B01D 71/10; B01D 67/00; C07C 29/80

(52) U.S. Cl. CPC B01D 71/70; B01D 2323/30; C08L 83/04; C08J 5/18; C08J 9/0061; B01D 61/027; B01D 61/362; B01D 71/10; B01D 67/0006; C07C 29/80; C08J 2301/02; C08J 2383/04; C08J 2401/02; C08J 2483/04; C08L 1/02

(57) ABSTRACT

Embodiments of the present disclosure describe polymer blend membranes comprising a layer including a polymer blend of regenerated cellulose and polydimethylsiloxane and a support in contact with the layer. Embodiments of the present disclosure describe methods of preparing a polymer blend membrane comprising contacting a cellulose precursor and a PDMS precursor in a solvent to form a polymer blend solution, depositing the polymer blend solution on a surface of a suitable support, curing the PDMS precursor of the polymer blend solution to form PDMS, and converting the cellulose precursor to cellulose to form a polymer blend membrane including cellulose and PDMS. Embodiments of the present disclosure describe methods of separating chemical species by one or more of organic solvent nanofiltration and pervaporation.

### BACKGROUND

Organic solvents are immensely important in an evolving range of industrial applications such as pharmaceutical, fine chemicals, petrochemicals, food, and electronics. The global solvent market reached US$25 billion in 2013 and is expected to increase by 4% per year until 2021. Membrane technology is emerging as a viable process for molecular separation in various media including organic solvents. Compared to the conventional distillation process, membrane technology is attractive because it is cost-effective, generates a smaller footprint, and can be operated in a much simpler way. It is projected to consume up to 90% less energy than that currently used for distillation. In particular, organic solvent nanofiltration (OSN) and pervaporation are developed for solvent separation and purification driven solely by a pressure gradient across the membrane. Chemically stable membranes with high flux and selectivity are necessary for a successful solvent processing. Despite the significant progress made in the last decade, it is still challenging to fabricate high performance membranes from low-cost materials through simple processes.

### SUMMARY

In general, embodiments of the present disclosure describe polymer blend membranes comprising cellulose and polydimethylsiloxane (PDMS), methods of preparing polymer blend membranes, methods of separating chemical species by organic solvent nanofiltration, methods of separating chemical species by pervaporation, and the like.

Embodiments of the present disclosure describe a method of preparing a polymer blend membrane comprising contacting a cellulose precursor and a PDMS precursor in a solvent to form a polymer blend solution, depositing the polymer blend solution on a surface of a suitable support, curing the PDMS precursor of the polymer blend solution to form PDMS, and converting the cellulose precursor to cellulose to form a polymer blend membrane including cellulose and PDMS.

Embodiments of the present disclosure describe a polymer blend membrane comprising a layer including a polymer blend of regenerated cellulose and PDMS, and a support in contact with the layer.

Embodiments of the present disclosure describe methods of separating chemical species by organic solvent nanofiltration contacting a first side of a polymer blend membrane with a feed stream containing one or more solutes dissolved in an organic solvent; and collecting a permeate from a second side of the membrane.

Embodiments of the present disclosure describe methods of separating chemical species by pervaporation contacting a first side of a polymer blend membrane with a liquid feed stream containing one or more solvents; and collecting a permeate in a vapor or gas phase from a second side of the membrane.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a flowchart of a method of preparing a polymer blend membrane, according to one or more embodiments of the present disclosure.

FIG. 2 is a flowchart of a method of separating chemical species by organic solvent nanofiltration, according to one or more embodiments of the present disclosure.

FIG. 3 is a flowchart of a method of separating chemical species by pervaporation, according to one or more embodiments of the present disclosure.

FIGS. 4A-4B are schematic diagrams illustrating water and ethanol diffusion paths in a PDMS and PDMS-RC blend membrane, according to one or more embodiments of the present disclosure.

FIG. 5 is a schematic diagram of a method of preparing cellulose-PDMS blend composite membranes on a PAN support, according to one or more embodiments of the present disclosure.

FIG. 6 is an image of a pervaporation setup, according to one or more embodiments of the present disclosure.

FIG. 7 is a schematic diagram of a pervaporation setup, according to one or more embodiments of the present disclosure.

FIGS. 8A-8E illustrate the changes of the 50RC properties before and after vapor-phase hydrolysis (VPH): A. ATR-IR spectra using the bare PAN support as a background, B. XPS survey composition, C. EDX silicon map and D. EDX silicon line scan, and E. the corresponding static water contact angle, according to one or more embodiments of the present disclosure.

FIGS. 9A-9F illustrate membrane chemistry: A. ATR-IR spectra and B. XPS atomic composition of different composition of the blend membranes. C. XPS survey of the 50RC membrane revealing the curve-fitting in the D. C1s, E. Si2p, and F. O1s region, according to one or more embodiments of the present disclosure.

FIGS. 10A-10F illustrate membrane surface properties: A. EDX silicon map and the corresponding static water contact angle of different composition of the blend membranes, B-C. cross-sectional and D. surface SEM images, E. AFM contrast-phase and F. topographic image of the 50RC membrane, according to one or more embodiments of the present disclosure.

FIGS. 11A-11F illustrate OSN performance: A. Rejection and B. flux in methanol, C. rejection and D. flux in hexane, E. pure solvent permeances, and F. the effects of compaction during methanol and hexane filtration on the 50RC membrane (feed pressure in experiment A-E: 8 bar), according to one or more embodiments of the present disclosure.

FIGS. 12A-12C illustrate pervaporation performance of different composition of the blend membranes: A. 5 wt % ethanol/water feed at 24° C. and 10 wt % ethanol/water feed at B. 24° C. and C. 60° C., according to one or more embodiments of the present disclosure.

### DETAILED DESCRIPTION

The invention of the present disclosure relates to polymer blend membranes, methods of making polymer blend membranes, and methods of using the membranes for various separation applications, such as organic solvent nanofiltration (OSN) and pervaporation. Surprisingly, the membranes of the present disclosure may include polymer blends of cellulose and polydimethylsiloxane (PDMS), despite each of those components having opposite properties. In this way, the membranes may combine (e.g., synergistically combine) the advantages of each of cellulose and PDMS to provide a membrane with enhanced separation performance (e.g., the selectivity of cellulose and/or flexibility of PDMS) for organic solvent nanofiltration and pervaporation, among other things. For example, through a combination of the rubbery PDMS and rigid cellulose structure, the polymer blend membranes may overcome the selectivity-permeability tradeoff in OSN, while the created amphiphilic feature may improve PDMS selectivity in, for example, ethanol-water pervaporation.

The polymer blend membranes of the present disclosure may be prepared by transforming cellulose into a cellulose precursor by functionalization. The resulting cellulose precursor may be solution processable such that it may be combined with PDMS in a wide array of solvents, such as organic solvents, to form highly compatible polymer blend solutions for use in the fabrication of membranes based on cellulose and PDMS. For example, the polymer blend solutions may be deposited on a suitable support and cured to form a well-integrated network of the cellulose precursor and the PDMS. The cellulose precursor may be regenerated in situ to cellulose after curing to achieve a cellulose/PDMS blend layer of a polymer blend membrane. The polymer blend membranes may remain homogenous before and/or after cellulose regeneration.

The invention of the present disclosure also relates to methods of separating chemical species by, for example, organic solvent nanofiltration and/or pervaporation. The interplay between the two polymers—one hydrophilic and rigid, the other one hydrophobic and flexible—provide membranes with significantly enhanced separation performance when compared with the pure polymer membranes. For example, in the case of organic solvent nanofiltration, the cellulose present in the interpenetrating PDMS/cellulose network reduced the swelling of the PDMS by the organic solvent and increased the membranes selectivity without large flux reduction. Due to the nature of the blend, the membranes may be used with polar and non-polar solvents. In the case of pervaporation of water/ethanol mixtures the addition of the water selective cellulose to PDMS surprisingly resulted in blend membranes with significantly enhanced ethanol separation ability. The presence of the cellulose in the matrix forced water through slower diffusion paths and in comparison with pure PDMS, water diffusivity was reduced and ethanol/water separation factor enhanced.

### Definitions

The terms recited below have been defined as described below. All other terms and phrases in this disclosure shall be construed according to their ordinary meaning as understood by one of skill in the art.

As used herein, “PDMS” refers to polydimethylsiloxane, including derivatives of polydimethylsiloxane.

As used herein, “polymer blend” refers to any mixture of two or more polymers. A “polymer blend” may be provided in any phase, such as a gel, liquid, solid, gas, vapor, or combination thereof. For example, the term “polymer blend” may describe a membrane or a solution. In general, a polymer blend may be compatible and either immiscible or miscible, or incompatible. For example, a compatible polymer blend generally includes a mixture of two or more polymers that is homogeneous on a macroscopic scale. A compatible polymer blend that is miscible generally includes a mixture of two or more polymers that is homogenous on both a microscopic scale and macroscopic scale. A compatible polymer blend that is immiscible generally includes a mixture of two or more polymers that is heterogeneous on a microscopic scale and homogenous on a macroscopic scale. An incompatible polymer blend generally includes a mixture of two or more polymers that is heterogeneous on both a microscopic scale and macroscopic scale.

As used herein, “molecular weight cutoff” or “MWCO” refers to the molecular weight of a molecule that is 90% retained by the membrane.

As used herein, “collecting” refers to recovering a component, such as a permeate or retentate. The component may include or be a product and one or more other chemical species. The product may also be an isolated product without any impurities, with a low concentration of impurities, or with a negligible concentration of impurities.

As used herein, “contacting” refers to the act of touching, making contact, or of bringing to close or immediate proximity, including at the cellular or molecular level, for example, to bring about a physiological reaction, a chemical reaction, or a physical change (e.g., in solution, in a reaction mixture, in vitro, or in vivo). Contacting may refer to bringing two or more components in proximity, such as physically, chemically, electrically, or some combination thereof. In addition or in the alternative, the contacting may include one or more of feeding, flowing, passing, injecting, introducing, and/or providing the fluid composition (e.g., a feed gas).

As used herein, “converting” refers to any process for producing cellulose from a cellulose precursor. For example, the “converting” may include restoring and/or regenerating cellulose from a cellulose precursor.

As used herein, “curing” refers to any process by which a material is transformed such that the transformed material exhibits one or more properties that are different from the original, non-transformed material. For example, curing may include, but is not limited to, thermal curing processes, such as heating to a specific temperature or within a specific temperature range; exposure to electromagnetic radiation (e.g., photocuring processes), such as exposure to electromagnetic radiation of a specific wavelength or wavelength range (e.g., ultraviolet electromagnetic radiation); exposure to gamma rays and/or electron beams; temporal curing processes, such as waiting for a specified time or time duration; drying processes, such as removal of all or a percentage of water or other solvent molecules; and any combination of these. Upon curing, a liquid or gel material may become solid or rigid and/or a pre-polymer material may transform into a polymer material. Non-limiting examples of curing include thermal curing, chemical curing, catalyst curing, radiation curing, and physical curing.

As used herein, “depositing” refers to disposing a material on, in, or around an object or mold, among other things. For example, depositing may refer to disposing a solution of on a substrate and/or support. Depositing may include, but is not limited to, one or more of spin-coating, coating, spreading, casting, applying, and pouring.

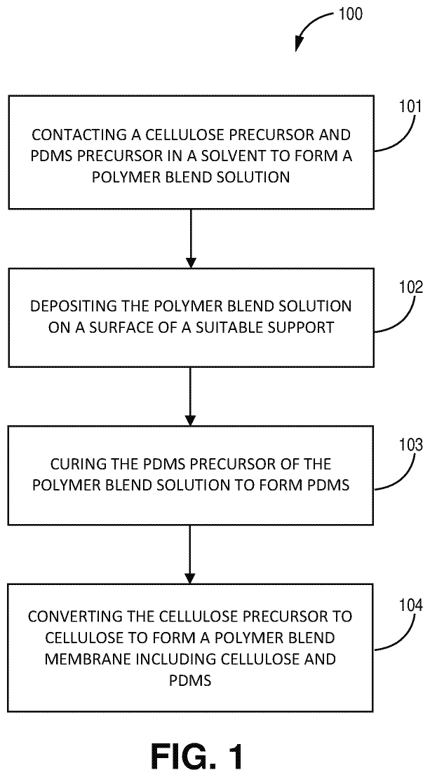

FIG. 1 is a flowchart of a method of preparing a polymer blend membrane, according to one or more embodiments of the present disclosure. As shown in FIG. 1, the method 100 may comprise contacting 101 a cellulose precursor and a PDMS precursor in a solvent to form a polymer blend solution, depositing 102 the polymer blend solution on a surface of a suitable support, curing 103 the PDMS precursor of the polymer blend solution to form PDMS, and converting 104 the cellulose precursor to cellulose to form a polymer blend membrane including cellulose and PDMS. An optional step (not shown) may include functionalizing a cellulose compound to provide a cellulose precursor.

The step 101 includes contacting a cellulose precursor and a PDMS precursor in a solvent to form a polymer blend solution. In this step, the cellulose precursor, PDMS precursor, and solvent may be brought into physical contact, or immediate or close proximity. The contacting may proceed by one or more of dissolving, mixing, and adding, among other forms of contacting. For example, in many embodiments, the cellulose precursor and PDMS precursor may be one or more of dissolved in, mixed in, and added to the solvent. The contacting of the cellulose precursor, PDMS precursor, and solvent may form the polymer blend solution.

A suitable cellulose precursor may include cellulose and/or derivatives thereof, such as functionalized cellulose, with one or more of the following characteristics: a cellulose precursor with one or more properties (e.g., chemical properties) that are similar to the properties of one or more of the PDMS precursor and the PDMS; a cellulose precursor that is compatible with one or more of the PDMS precursor and the PDMS; a cellulose precursor that is capable of forming a polymer blend with one or more of the PDMS precursor and the PDMS; a cellulose precursor that is capable of being converted or regenerated to cellulose; and a cellulose precursor that is processable in solvents, such as common organic solvents. In many embodiments, cellulose may be modified and/or functionalized to produce a cellulose precursor with one or more of the characteristics described above.

In an embodiment, cellulose may be functionalized to form an amorphous hydrocarbon-soluble polymer. In an embodiment, the cellulose may be functionalized with silylating agents. Examples of suitable silylating agents include, but are not limited to, trimethylsilyl (TMS), dimethylsilyl (DMS), triethylsilyl (TES), triisopropylsilyl (TIPS), t-butyldimethylsilyl (TBS/TBDMS), and t-butyldiphenylsilyl (TBDPS). For example, in an embodiment, the cellulose precursor is functionalized with trimethylsilyl, wherein the cellulose precursor includes a cellulose containing one or more trimethylsilyl groups per glucose unit. The TMSC may be a preferred cellulose precursor for any of numerous reasons. The TMSC is a hydrophobic cellulose derivative that may be easily synthesized. The TMSC has high processability in many organic solvents and good miscibility with PDMS in the polymer blend solution. The TMSC may be blended with PDMS to form a compatible polymer mixture in commonly used organic solvents. The TMSC may also be regenerated to cellulose as described herein. While TMSC is described herein, TMSC shall not be limiting, as other cellulose precursors may be used herein.

The PDMS precursor may include a pre-polymer and a crosslinker. For example, in an embodiment, the PDMS precursor may include vinyl-terminated dimethyl siloxane as the pre-polymer and/or hydrosilane crosslinker as the crosslinker. In other embodiments, the PDMS or its precursor (e.g., prepolymer) can also be functionalized with end groups other than vinyl. Examples of suitable end groups include, but are not limited to, hydride (—H), methyl (—CH₃), 3-aminopropyl (—CH₂CH₂CH₂NH₂), glycidyl ether (—CH₂CH₂CH₂OCH₂), hydroxyl (—OH), alkyhydroxy (—(CH₂)ₘOH), chloride (—Cl), and/or stearate

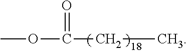

In some embodiments, the PDMS or its precursor can be presented as a copolymer with functional groups that include, but are not limited to, hydride (—H), 3-(2-(2-hydroxyethoxy)ethoxy)propyl (—CH₂CH₂CH₂(OCH₂CH₂)₂OH), poly(ethylene glycol) methyl ether (—CH₂CH₂CH₂O(CH₂CH₂O), CH₃), 3-aminopropyl, —CH₂CH₂CH₂NH₂, and polyacrylates. The PDMS precursor is not particularly limiting and can include any precursors suitable for forming one or more of the PDMS and derivatives thereof.

The solvent may include one or more of water and an organic solvent. In many embodiments, the solvent includes an organic solvent, such as common organic solvents. The organic solvent may include one or more of polar organic solvents, non-polar organic solvents, protic organic solvents, and aprotic organic solvents. The organic solvent may include one or more of aromatic compounds, alcohols, esters, ethers, ketones, amines, and hydrocarbons, including nitrated hydrocarbons and/or halogenated hydrocarbons. Examples of organic solvents may include, but are not limited to, one or more of hexane, octane, acetone, tetrahydrofuran (THF), dimethylpiperidone (DMPD), N-methylpyrrolidinone (NMP), and dimethylacetamide (DMAc), dimethylformamide (DMF), methanol, ethanol, isopropanol, ethylene glycol, methylene chloride, ethyl acetate, dimethyl sulfoxide, benzene, toluene, and xylene, among others.

The polymer blend solution may be characterized as a polymer blend. In many embodiments, the polymer blend solution is homogenous on a macroscopic scale (e.g., a compatible polymer blend). In preferred embodiments, the polymer blend solution is homogenous on a macroscopic scale and a microscopic scale (e.g., a miscible polymer blend). For example, the polymer blend solution may be one or more of transparent, exhibit no visible phase separation. In other embodiments, the polymer blend solution is homogenous on a macroscopic scale and heterogeneous on a microscopic scale (e.g., an immiscible polymer blend).

The step 102 includes depositing the polymer blend solution on a surface of a suitable support. In this step, the polymer blend solution is deposited on the suitable support by one or more of one or more of spin-coating, coating, spreading, casting, applying, and pouring. The polymer blend solution may be deposited in the form of a layer or film (e.g., thin film), among other forms, on the support. The amount of the solution coating and technique used to deposit the solution coating may be varied to produce a polymer blend membrane with varying thicknesses. The amount or technique used can be selected to, for example, reduce the presence of unselective pin holes and/or maintain a desired permeation flux, as thicknesses that are too thin can be defective and thicknesses that are too high can significantly decrease the permeation flux. In an embodiment, the thickness of the polymer blend solution or polymer blend membrane can range from a few tenths of nanometer to around 150 nm. In an embodiment, the thickness of the polymer blend solution or the polymer blend membrane is greater than about 10 nm and less than about 150 nm.

The support may include any suitable support. The support may include one or more of an organic material and an inorganic material. In many embodiments, the support includes a polymer material. Examples of suitable polymers for the support include, but are not limited to, one or more of polyetherimides; polyolefins including polyethylene and polypropylene, as well as copolymers thereof; polysulfones such as aromatic polysulfones, polyethersulfones, etc.; halopolymers such as polyvinyl chloride, polyvinylidene chloride, polyvinylidene fluoride, etc.; polyesters such as polyethyleneterephthalate (PET), polytrimethylene terephthalate, polybutylene terephthalate, etc.; polyamides such as nylon 6, nylon 66, nylon 612, nylon 12, etc., aromatic polyamides; polycarbonates; polystyrenes; polyacrylonitriles; polyacrylates such as polymethylmethacrylate, copolymers of acrylic acid, methacrylic acid, hydroxyethylmethacrylate, etc.; polyacetates such as polyvinyl acetate and partially hydrolyzed polyvinyl acetates; polyalcohols such as polyvinyl alcohol, cationically modified polyvinylalcohol, anionically modified polyvinylalcohol; polysaccharides such as chitosan, hyaluronan, cellulose, regenerated cellulose, cellulose ethers such as methylcellulose, ethylcellulose, hydroxyethyl cellulose, cellulose esters such as cellulose acetates (including mono-, di-, and tri-acetates); proteins such as collagen, gelatin, etc.; ionomers; polyalkylene oxides such as polyethylene oxide, polypropylene oxide, polyethylene glycols, crosslinkable polyethylene glycol, etc.; polyurethanes; polyureas; poly(urethane-urea); polyimines such as polyethylene imine; polyvinylpyrrolidone; polyacrylic acids; polymethacrylic acids; polysiloxanes such as polydimethylsiloxane; poly(ester-co-glycol) copolymers; poly(ether-co-amide) copolymers; and mixtures, derivatives, copolymers and crosslinked forms of any of the above. Derivatives include ethers, esters, amides, etc. formed by alkylation, acylation etc. of functional groups (e.g., hydroxyl or amine groups), or by hydrolysis of hydrolyzable functional groups (e.g., esters, amides, anhydrides, etc.) present in the polymer of which the nanofiber is comprised. In many embodiments, the support includes one or more of polyacrylonitrile (PAN), polyethersulfone (PES), polyvinylidenefluoride (PVDF), crosslinked water-soluble polymers such as polyvinyl alcohol, modified cellulose, modified chitosan, etc. In other embodiments, the support may include metal oxides.

The step 103 includes curing the PDMS precursor of the polymer blend solution to form PDMS. In this step, the deposited polymer blend solution may be subjected to conditions sufficient to crosslink and form the PDMS. In many embodiments, the curing may include subjecting the polymer blend solution and/or support to heat or an environment in which a temperature is increased. For example, the curing may be achieved by heating in an oven at a select temperature for a select duration. The select temperature may include any temperature suitable for curing. For example, the select temperature may range from about 25° C. to about 150° C. In an embodiment, the select temperature is about 100° C. The select duration may include any duration suitable for curing. For example, the select duration may range from about 10 min to about 48 h. In an embodiment, the select duration is about 2 h. In other embodiments, the curing may be achieved by any process or technique for crosslinking/curing the PDMS, such as exposing to UV.

The curing may initiate a crosslinking reaction whereby the pre-polymer of the PDMS precursor is crosslinked by the crosslinker of the PDMS precursor to form the PDMS. For example, in an embodiment, the PDMS may be formed through catalytic hydrosilylation between a vinyl-terminated dimethylsiloxane and a hydrosilane-containing crosslinker. In many embodiments, the PDMS formation may proceed in the presence of the cellulose precursor, such that the curing forms a membrane including a well-interpenetrating network of the PDMS and the cellulose precursor. The membrane including the PDMS and the cellulose precursor may not exhibit any sign of polymer segregation or at least may not exhibit any significant polymer segregation. The membrane including the PDMS and the cellulose precursor may exhibit good homogeneity.

The step 104 includes converting the cellulose precursor to cellulose to form a polymer blend membrane including cellulose and PDMS. In this step, cellulose is regenerated from the cellulose precursor to form a regenerated cellulose-PDMS polymer blend membrane. The converting may include any process or technique suitable for regenerating cellulose, preferably without degrading (e.g., depolymerizing) the PDMS. In many embodiments, the converting may depend on the functional group, chemical group, or moiety used to functionalize/modify the cellulose and form the cellulose precursor. For example, in an embodiment (e.g., such as embodiments where the cellulose precursor is or includes TMSC), the converting may include vapor-phase hydrolysis using an acid, such as hydrochloric acid. The acid vapor may preferentially attack the silyl group of the TMSC, as opposed to the siloxane positioned on the PDMS backbone, sufficient to remove the silyl groups therefrom and restore the hydroxyl groups previously present in the cellulose. In an embodiment, cellulose regeneration from TMSC can be readily performed by reintroducing the active hydrogen. In an embodiment, cellulose regeneration can also be achieved by immersion in 1M hydrochloric acid solution, which may not preferable for membrane manufacture as it can create defects easily. In an embodiment, cellulose regeneration can also be achieved by hydrolysis with boiling water and simple alcohols (e.g., ethanol, propanol, isopropanol, n-butanol, and other C1-C20 alcohols). The converting may completely, substantially, or partially restore the cellulose. In an embodiment, the converting may completely restore the cellulose such that the functional groups of the cellulose precursor are undetectable or negligible. In an embodiment, the converting may substantially restore the cellulose such that the functional groups of the cellulose precursor are minimally detectable. In an embodiment, the converting may partially restore the cellulose such that the functional groups of the cellulose precursor are present in any amount (e.g., more than a negligible amount).

In an embodiment, the method of preparing a membrane may comprise contacting trimethylsilyl cellulose and polydimethylsiloxane in an organic solvent to form a coating solution, depositing the coating solution on a polyacrylonitrile support to form a coated support, curing the coated support to form a blend membrane including the trimethylsilyl cellulose and crosslinked polydimethylsiloxane, and regenerating the trimethylsilyl cellulose of the blend membrane to cellulose by vapor-phase hydrolysis.

Embodiments of the present disclosure describe polymer blend membranes. The polymer blend membranes may comprise a layer including a polymer blend and a support in contact with the layer. The polymer blend membrane may include any of the polymer blends and supports of the present disclosure. For example, the polymer blend may include one or more of a cellulose, cellulose precursor, and regenerated cellulose. The support may include a polymeric support. In many embodiments, the polymer blend membranes comprise a layer including a polymer blend of regenerated cellulose and PDMS, and a support in contact with the layer.

In many embodiments, the polymer blend includes regenerated cellulose. The regenerated cellulose may include any cellulose converted or restored from a cellulose precursor, such as a cellulose derivative. For example, a regenerated cellulose may include any cellulose precursor that has been completely, substantially, or partially restored to cellulose. The cellulose precursor may be restored to cellulose by, for example, removal of the functional groups of the cellulose precursor using any of the methods, processes, or techniques described herein or known in the art. In a completely restored cellulose, the functional groups of the cellulose precursor may be undetectable or negligible. In a substantially restored cellulose, the functional groups of the cellulose precursor may be minimally detectable. In a partially restored cellulose precursor, the functional groups of the unrestored cellulose precursor may be present in any amount.

The polymer blend membranes may be thin-film composite membranes. For example, the layer including the polymer blend of the cellulose and PDMS may be provided as a film or thin film on a surface of the support. The layer may be fabricated independently of the support to allow for optimization of the layer. The layer may be characterized by a highly smooth layer without or substantially without any observable defects. A thickness of the layer of the polymer blend membrane may range from about 1 nm to about 10 μm. In many embodiments, a thickness of the layer of polymer blend membrane may range from about 10 nm to about 100 nm. In a preferred embodiment, a thickness of the layer of the polymer blend membrane may be about 40 nm

The polymer blend membranes may include a total polymer concentration ranging from about greater than 0 wt % to about 50 wt %. In many embodiments, a total polymer concentration of the polymer blend membranes is less than about 10 wt %. In preferred embodiments, a total polymer concentration of the polymer blend membranes ranges from about 1 wt % to about 4 wt %, or more preferably about 2 wt %. The cellulose may range from greater than about 0% to about 100% of the total polymer content, with the balance being the PDMS. For example, in an embodiment, the cellulose may be about 25% of the total polymer content. In an embodiment, the cellulose may be about 50% of the total polymer content. In an embodiment, the cellulose may be about 75% of the total polymer content.

Surface properties of the polymer blend membranes may vary depending on the concentration of one or more of the regenerated cellulose and PDMS. For example, the cellulose or regenerated cellulose may independently be characterized as hydrophilic (e.g., strongly hydrophilic), whereas PDMS may independently be characterized as hydrophobic. A surface property of the polymer blend membrane, which combines the regenerated cellulose and PDMS into a well-integrated network or matrix, may be tuned by varying the concentration of the regenerated cellulose and PDMS. The surface property may depend on the total concentration of polymers (e.g., cellulose and PDMS) and/or on the ratio of the cellulose to PDMS. For example, a hydrophilicity of the polymer blend membrane may increase as a concentration of the cellulose increases and/or a concentration of the PDMS decreases. A hydrophobicity of the polymer blend membrane may increase as a concentration of the PDMS increases and/or as a concentration of the cellulose decreases.

The polymer blend membranes combine a selectivity of the cellulose with the flexibility of the PDMS to achieve chemically stable membranes with high flux and rejection. The incorporation of cellulose into the PDMS matrix may reduce the PDMS chain flexibility to improve molecular sieving. The PDMS in the cellulose network may interrupt the densely packed cellulose structure to promote higher solvent fluxes (e.g., especially in non-polar solvents such as hexane). The highly polar and highly non-polar segments of the polymer blend membrane may allow the easy passage of solvent with any polarity.

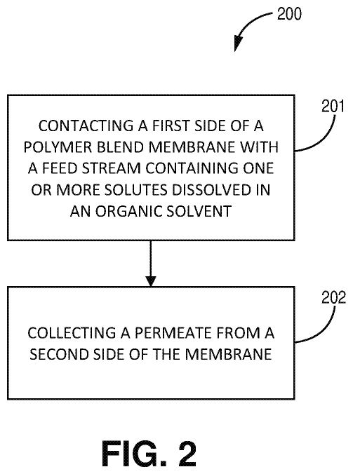

FIG. 2 is a flowchart of a method of separating chemical species by organic solvent nanofiltration, according to one or more embodiments of the present disclosure. As shown in FIG. 2, the method 200 may comprise contacting 201 a first side of a polymer blend membrane with a feed stream containing one or more solutes dissolved in an organic solvent and collecting 202 a permeate from a second side of the polymer blend membrane.

The step 201 includes contacting a first side of a polymer blend membrane with a feed stream containing one or more solutes dissolved in an organic solvent. In this step, the feed stream is fed, usually under pressure, sufficient to make contact with the first side of the polymer blend membrane and produce a permeate and a retentate. The permeate may include the solvent (e.g., organic solvent) and solute molecules (e.g., small solute molecules) that pass through or permeate the membrane. The retentate may include the solvent and other solute molecules (e.g., large solute molecules) that are retained or rejected by the membrane (e.g., do not permeate the membrane).

The contacting may include feeding, flowing, and/or passing the feed stream, among other types of contacting. The contacting may proceed at a pressure ranging from about 2 bar to about 20 bar. In other embodiments, the contacting may proceed at a pressure less than about 2 bar and/or greater than about 20 bar. In many embodiments, the contacting may proceed at a pressure over which compaction is minimized and/or not observed.

The polymer blend membranes may be used in organic solvent nanofiltration for the separation of molecules in the range of about 200 to about 1000 Da. The polymer blend membranes may be used in any solvent, regardless of polarity, for solvent separations, solute/solvent separations, etc. For example, the polymer blend membranes may be used in highly polar solvents, such as methanol, as well as in non-polar solvents, such as hexane. The highly polar and highly non-polar segments of the polymer blend membrane may permit passage of the solvent with any polarity. Permeance may range from about 0.1 L/m² h bar to about 15 L/m² h bar. For example, in many embodiments, the permeance may be greater than about 10 L/m² h bar. A molecular weight cutoff of the polymer blend membranes may range from about 500 Da to about 1000 Da. For example, in many embodiments, a molecular weight cutoff may be about 750 Da.

The first side of the polymer blend membrane may include a polymer blend layer of the polymer blend membrane. The polymer blend layer may include a well-integrated network of cellulose (e.g., regenerated cellulose) and PDMS. A total polymer concentration of the polymer blend layer may range from about greater than 0 wt % to about 50 wt %. In many embodiments, a total polymer concentration of the polymer blend layer may be about 2 wt %. A concentration of the cellulose (e.g., regenerated cellulose) may range from about greater than 0 wt % to about less than 100 wt %, with the balance being PDMS. In many embodiments, a concentration of the cellulose may range from about 25 wt % to about 75 wt %. In preferred embodiments, a concentration of the cellulose is about 50 wt %.

The feed stream may include one or more solutes dissolved and/or present in a solvent. The solvent may include any organic solvent. For example, the organic solvent may include one or more of isopropanol, ethanol, acetonitrile, tetrahydrofuran, toluene, chloroform, hexane, methanol, acetone, dimethyl sulfoxide (DMSO), dimethylformamide (DMF), n-methyl 2-pyrrolidone (NMP), 1-propanol, 2-propanol, 1-butanol, 1-hexanol, 1-octanol, trifluoroethanol, propylene glycol, PEG 400, 1,3-propanediol, diethyl ether, diglyme, decalin, isooctane, mineral oil, benzene, chlorobenzene, pyridine, ethyl acetate, methyl acetate, dichloroethane, ethylene diamine, and trimethyl phosphate.

The step 202 includes collecting a permeate from a second side of the membrane. In this step, the solvent and solute molecules that passed through or permeated the membrane are collected as the permeate. In some embodiments, the retentate may further be collected and recycled in the process. For example, in an embodiment, the retentate may be collected and re-contacted with the membrane one or more times.

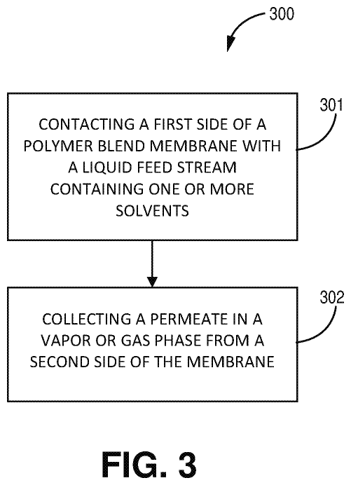

FIG. 3 is a flowchart of a method of separating chemical species by pervaporation, according to one or more embodiments of the present disclosure. As shown in FIG. 3, the method may comprise contacting 301 a first side of a polymer blend membrane with a liquid feed stream containing one or more solvents and collecting 302 a permeate in a vapor or gas phase from a second side of the membrane.

The step 301 includes contacting a first side of a polymer blend membrane with a liquid feed stream containing one or more solvents. In this step, the liquid feed stream is fed, flowed, and/or passed sufficient to make contact with the first side of the membrane and produce a vaporous permeate and a retentate. The permeate may be permeated through the membrane and evaporated to produce the vaporous permeate. Transport through the membrane may be driven by a difference in partial pressure between the liquid feed stream and the permeate. In many embodiments, a vacuum is applied to provide the partial pressure difference driving force. In other embodiments, an inert gas may be applied to provide the partial pressure difference driving force.

The polymer blend membrane may be used for solvent/solvent separations, solute/solvent separations, etc. In many embodiments, the polymer blend membrane may be used for traditionally difficult separations, such as azeotropic separations, which generally require high capital costs and high energy costs. For example, the polymer blend membranes may be used for the separation of ethanol-water streams, such a water-rich ethanol-water streams and/or ethanol-rich ethanol-water streams. The blending of cellulose (e.g., regenerated cellulose) and PDMS into a polymer blend membrane may enhance membrane performance. For example, in an ethanol/water separation, the separation factor may be about 14 and the flux may be about 1.6 kg/m² h. In this way, the polymer blend membranes may facilitate the selective ethanol transport while maintaining the exceptionally high flux of PDMS.

The first side of the polymer blend membrane may include a polymer blend layer of the polymer blend membrane. The polymer blend layer may include a well-integrated network of cellulose (e.g., regenerated cellulose) and PDMS. A total polymer concentration of the polymer blend layer may be about 2 wt %. A concentration of the cellulose (e.g., regenerated cellulose) may range from about greater than 0 wt % to about less than 100 wt %, with the balance being PDMS. In many embodiments, a concentration of the cellulose may range from about 25 wt % to about 75 wt %. In preferred embodiments, a concentration of the cellulose may be about 50 wt %.

The feed stream may include one or more solvents. The solvent may include one or more of water and any organic solvent. In many embodiments, the feed stream may include ethanol and water. In other embodiments, the feed stream may include one or more of water, dimethyl sulfoxide (DMSO), dimethylformamide (DMF), tetrahydrofuran (THF), methanol, ethanol, toluene, n-methyl 2-pyrrolidone (NMP), acetone, 1-propanol, 2-propanol, 1-butanol, 1-hexanol, 1-octanol, trifluoroethanol, propylene glycol, PEG 400, 1,3-propanediol, diethyl ether, diglyme, decalin, isooctane, mineral oil, benzene, chlorobenzene, pyridine, ethyl acetate, methyl acetate, dichloroethane, ethylene diamine, acetonitrile, and trimethyl phosphate.

The step 302 includes collecting a permeate in a vapor or gas phase from a second side of the membrane. In this step, the components of the feed that passed through or permeated the membrane evaporate to form a permeate in a vapor or gas phase, such as a vaporous permeate. The collecting may include condensing the vaporous and/or gaseous permeate. In some embodiments, the retentate may further be collected and recycled in the process. For example, in an embodiment, the retentate may be collected and re-contacted with the membrane one or more times.

The following Examples are intended to illustrate the above invention and should not be construed as to narrow its scope. One skilled in the art will readily recognize that the Examiners suggest many other ways in which the invention could be practiced. It should be understand that numerous variations and modifications may be made while remaining within the scope of the invention.

### EXAMPLE 1

A novel method for the preparation of cellulose-polydimethylsiloxane (PDMS) blend membranes for organic solvent nanofiltration (OSN) and ethanol pervaporation is presented. The elegance of this approach relates to the use of trimethylsilyl cellulose (TMSC), a hydrophobic cellulose derivative, for the blend membrane fabrication, followed by a simple hydrolysis to convert the TMSC back into cellulose. The use of TMSC allows cellulose processing in common organic solvents and enables the creation of highly compatible cellulose-PDMS blend membranes. The proper composition of the blend membrane gave the best OSN performance with more than 10 L/m²hbar acetone permeance and around 750 Da molecular weight cut-off. The blend membrane can be used for OSN with any other solvents regardless of their polarity. The same membrane also displayed an excellent pervaporation performance with a separation factor of 14 and a flux of 1.6 kg/m²h flux using a 5 wt % ethanol-water mixture at room temperature. The blending of PDMS with cellulose resulted in a 100% increase of the separation factor when compared to pure PDMS. This surprising performance is a consequence of the good miscibility of the blending polymers, which can be obtained through this unique pathway. Overall, this Example demonstrates a simple, efficient, scalable and the only possible way reported to date to combine the superiority of cellulose and PDMS as a low-cost and stable membrane for solvent separation.

Polydimethylsiloxane (PDMS), often referred to as “silicon rubber”, emerged as an important membrane material with extensive uses in gas and liquid separations. It is a highly flexible polymer attributed to the free rotation of the Si—O bonds, making it highly processable and very permeable to numerous molecules. PDMS turns into a macromolecular mesh when swollen with non-polar solvents like hexane, compromising its selectivity. Various techniques have been developed to improve the separation properties of PDMS. In particular, incorporation of hydrophilic segments such as polyethylene oxide, polyethylene glycol, poly(methyl methacrylate), dopamine, and polyimide into PDMS have shown various degrees of selectivity improvement in gas separation and pervaporation, attributed either to the microphase-separated structure or the increasing chain rigidity.

Cellulose is another attractive polymer for membrane manufacturing due to its abundant availability, low cost, environment benignancy, and fascinating physiochemical properties. In particular, cellulose possess a unique molecular architecture, at which the repeating β-D-glucopyranose molecules are covalently bound through acetal linkages (β-1, 4-glucan), making it chemically resistant, inherently stiff and strongly hydrophilic. These properties endow cellulose as an excellent candidate for size exclusion carried out in solvent media, especially in polar solvents. However, its chemical stability also creates difficulties in fabricating cellulose membranes using the common organic solvents, advocating the use of the derivation-regeneration route as an alternative to the one-step fabrication using exotic or toxic solvents.

Intrigued by the remarkable properties of the aforementioned polymers while considering their limitations in the separation, cellulose-PDMS membranes were fabricated simply by physical blending. Compared to the complicated copolymerization approach commonly applied to modify PDMS, physical blending was more time- and cost-effective to modulate membrane performance by combining the superiority of the constituting polymers. Unfortunately, blending of these polymers was previously not possible due to their contrary properties. In the present Example, blending of cellulose and PDMS was realized by first modifying cellulose into trimethylsilyl cellulose (TMSC), followed by the in situ regeneration of TMSC back into cellulose after the blend membrane fabrication. TMSC is an easily synthesized cellulose derivative that has been used earlier to create microporous membranes for use in nanofiltration and water vapor/gas separation. Mixtures of PDMS and TMSC were dissolved in hexane without visible phase separation enabling the preparations of transparent films and coatings. The blends remained homogeneous even after cellulose regeneration. The fabricated membranes were investigated for molecular separation in organic solvents through organic solvent nanofiltration (OSN) and pervaporation. The combination of the rubbery PDMS and the rigid cellulose structure was expected to overcome the selectivity-permeability tradeoff in OSN, while the created amphiphilic feature is anticipated to improve the PDMS selectivity in ethanol-water pervaporation. Polyacrylonitrile, which is stable in a wide range of organic solvents, was used as a support for the thin film coating. The successful creation of the blend membranes was proved by many characterizations including differential scanning calorimetry, infrared spectroscopy, and elemental analysis. The changes of the membrane properties upon the cellulose regeneration were also investigated. Materials and Methods

Materials. TMSC was prepared from microcrystalline cellulose (Avicel PH101, Fluka) and was characterized. Dimethylacetamide (DMAc), lithium chloride, hydrochloric acid (HCl), n-hexane, methanol, acetone, tetrahydrofuran, ethanol, acetonitrile, chloroform, and toluene were purchased from Sigma Aldrich and used without further purification. Isopropanol was purchased from Acros Organics. MilliQ water was used in the pervaporation experiments. PDMS pre-mixed kit consisting of Sylgard 184A was used as a pre-polymer and Sylgard 184B was used as a crosslinker, which was obtained from Sewang Hitech Co. Ltd. (South Korea). Polyacrylonitrile (GMT GmbH, Germany) ultrafiltration membranes were used as supports. Dyes and polystyrene with different molecular weights for OSN experiments were purchased from Sigma Aldrich.

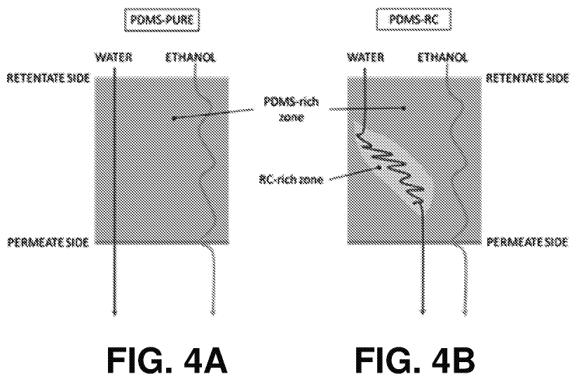

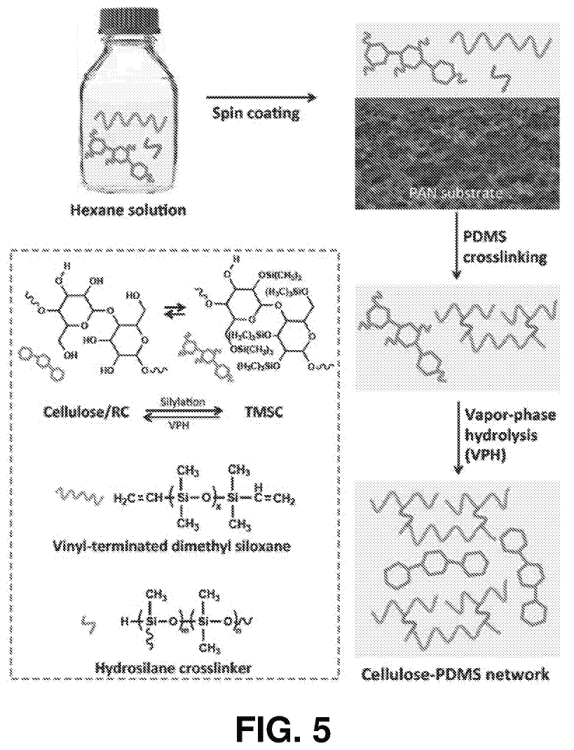

Membrane Preparation. Coating solutions were prepared by dissolving TMSC, PDMS, or mixture of both in n-hexane at room temperature. The composition of each membrane is listed in Table 1. A 10:1 ratio of pre-polymer to the crosslinker was used for PDMS formation, following the recommendation from the manufacturer. Polymer deposition on the PAN support was carried out by spin coating at a 4,000 rpm and 500 rpms⁻¹ speed and acceleration, respectively. TMSC-coated membranes were transformed back into cellulose through a vapor-phase hydrolysis (VPH) using a 10% HCl solution for 15 minutes (FIGS. 4A-4B), whereas pure PDMS membranes underwent curing at 100° C. for 2 h. In the case of the blend membranes, the coated supports experienced both curing and hydrolysis consecutively as illustrated in FIG. 5.

TABLE 1
Composition of the membranes prepared in this study.

| Membrane | TMSC (wt %) | PDMS (wt %) | Crosslinking | VPH |
|---|---|---|---|---|
| RC | 2 | — | — | 15 min |
| 75RC | 1.5 | 0.5 | 100° C., 2 h | 15 min |
| 50RC | 1 | 1 | 100° C., 2 h | 15 min |
| 25RC | 0.5 | 1.5 | 100° C., 2 h | 15 min |
| PDMS2 | — | 2 | 100° C., 2 h | — |
| PDMS4 | — | 4 | 100° C., 2 h | — |
| PDMS6 | — | 6 | 100° C., 2 h | — |

Membrane Characterization. The ATR-IR spectra of the membranes were collected using a Fischer Scientific Nicolet iS10 spectrometer and were recorded in the range between 500 and 4,000 cm over 16 scans. The bare PAN support was measured as a background. The XPS experiments were performed on a Kratos Axis Ultra DLD instrument equipped with a monochromatic Al Kα x-ray source (hv=1486.6 eV) operated at a power of 150 W and under UHV conditions in the range of 10⁻⁹ mbar. All spectra were recorded in hybrid mode using electrostatic and magnetic lenses and an aperture slot of 300 μm×700 μm. The survey and high resolution spectra were acquired at fixed analyzer pass energies of 160 eV and 20 eV, respectively. The samples were mounted in floating mode in order to avoid differential charging and XPS spectra were therefore acquired using charge neutralization. A Lorentzian-Gaussian function was used for the curve fitting on the C1s, Si2p and O1s region at their corresponding binding energies. Differential scanning calorimetry (DSC) was performed using a NETZSCH model DSC 204F1. About 5 mg samples were weighed in aluminum pans and analyzed with a heating rate of 10° C./min over a temperature range of −150° C.-350° C. Energy-dispersive X-ray (EDX) spectroscopy analysis was performed using an OXFORD EDX equipped with X-MaxN detector, an 80 mm² SDD sensor and AZtecEnergy analysis software. The accelerating voltage was 10 keV with a probe current of 5.5 nA. The membrane hydrophilicity was determined by a static water contact angle (WCA) measurement using a Kruess drop shape analyzer—DSA100 with monochrome interline CCD camera. Surface and cross-section SEM imaging was performed on the 3 nm iridium-coated membranes using a Teneo scanning electron microscopy at 3 kV with a working distance between 3-5 mm. The samples for cross-section imaging were fractured in liquid nitrogen. The surface topographies were analyzed by an atomic force microscopy (AFM) on an ICON Veeco microscope operating in the tapping mode using commercial silicon TM AFM tips (MPP 12100).

OSN Experiment. All nanofiltration experiments were carried out using a commercial HP4750 dead-end filtration cell (Sterlitech Corporation, USA) with an effective area of 13.85 cm² pressurized by nitrogen. Rejection experiments were carried out using either methanol or hexane containing neutral polystyrene and or neutrally charged dyes as markers (see neutral markers used for OSN experiments below).

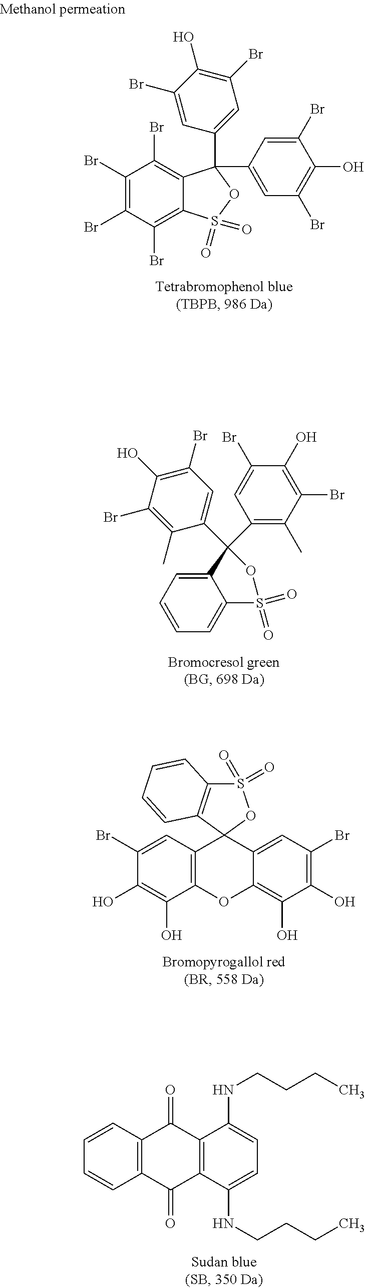

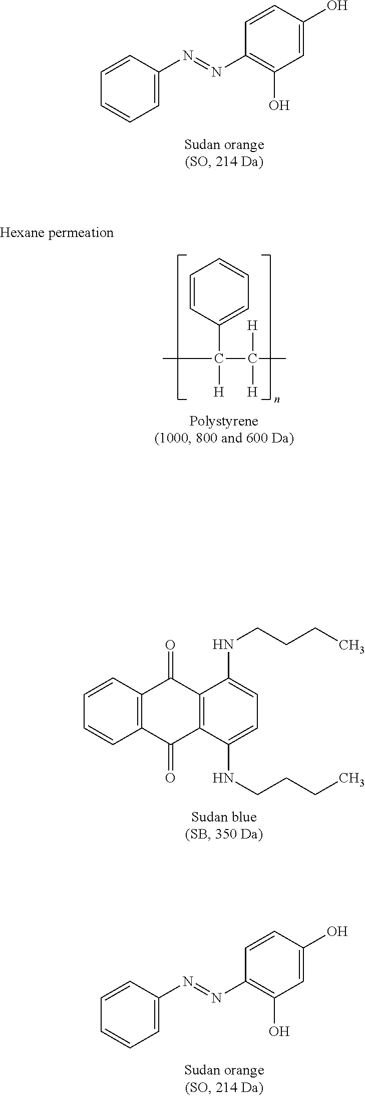

Permeances of the pure solvents were tested using methanol, hexane, ethanol, isopropanol, tetrahydrofuran (THF), toluene, chloroform, acetone, and acetonitrile. Prior to the measurements, the membranes were soaked in the respecting solvents for a minimum of 24 hours to reach equilibrium. The permeance, J_{w} (L/m²hbar), which is the pressure-normalized flux, was calculated using eq. (1):

$${J}_{w} = \frac{V}{A \times Δ t \times P}$$

where V is the volume of the permeate (L), A is the active membrane area (m²), Δt is the time for permeate collection (h), and P is the trans-membrane pressure (bar). Rejection value was determined as a ratio of permeate concentration over the averaged concentration of the initial and final feed solution following eq. (2). The rejection of dyes and polystyrene was monitored using a NanoDrop 2000/2000c spectrophotometer (Thermo Fisher Scientific).

$$Rejection ( \% ) = ( 1 - \frac{{C}_{p}}{{C}_{f}} ) \times 100$$

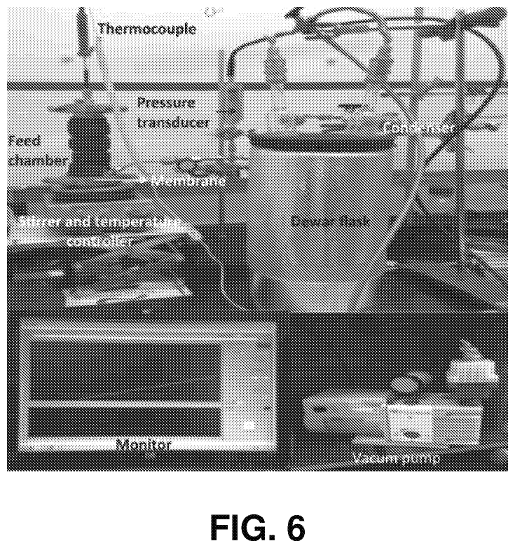

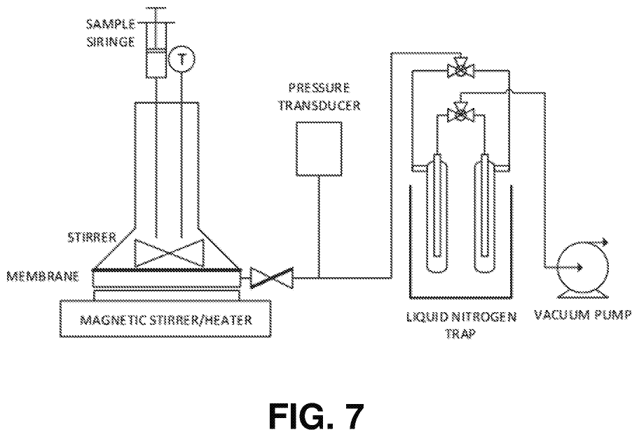

Pervaporation Experiment. FIGS. 6-7 show the in-house designed pervaporation unit. It is known that under turbulent flow it is possible to maximize feed side diffusion rate minimizing any polarization phenomena. Hence, a dead-end cell that allows mixing directly on top of the membrane was used, setting high stiffing rates (>400 rpm). The dead-end cell was modified for pervaporation, allowing sampling retentates and sensing temperatures directly at the stiffing zone (i.e., just above the membrane). The membrane active area was 11.33 cm². A Honeywell™ Super TJE of 14 PSIa pressure transducer was used to sense permeate pressure. Both temperature and transducer signals were recorded via a computer interface based on LABVIEW®. Two vacuum traps immersed in liquid nitrogen allowed collecting permeates. The combination of unit-configuration, pump installed, and membranes used permitted permeate-pressures even lower than 20 mATM.

The composition of the collected permeate was determined using an Agilent gas chromatography model 7890A with a flame ionization detector and DB-WAX column (30 m×320 μm×0.25 μm J & W 123-7032, 250° C.). The total flux, Jₚ (kg/m²h), was calculated according to eq. (3) by dividing the weight of the collected sample, m (kg), with the effective membrane area, A (m²), and time difference, Δt (h). The separation factor (SF), which is the basic parameter to measure the pervaporation performance, was calculated according to eq. (4), where X and Y are the weight fraction of species in the permeate and feed, respectively. Subscript a and b denote ethanol and water, respectively.

$${J}_{p} = \frac{m}{A \times Δ t}$$

$$SF = \frac{{( \frac{Xa}{Xb} )}_{p}}{{( \frac{Ya}{Yb} )}_{F}} = \frac{\frac{{( Xa )}_{p}}{{( 1 - Xa )}_{p}}}{\frac{{( Ya )}_{F}}{{( 1 - Ya )}_{F}}}$$

The pervaporation experiments were carried out using aqueous feed solutions containing 5 wt % and 10 wt % ethanol at 24° C. and 60° C. under atmospheric feed pressure and vacuum downstream.

### Results and Discussion

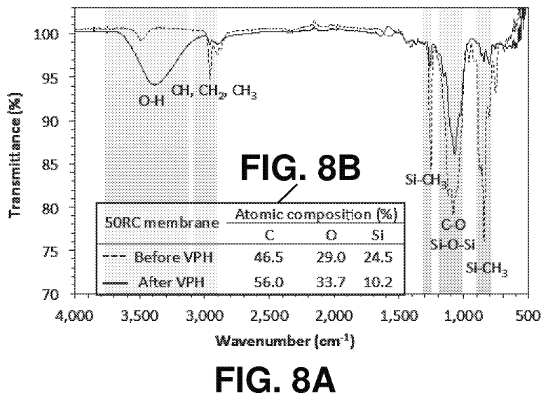

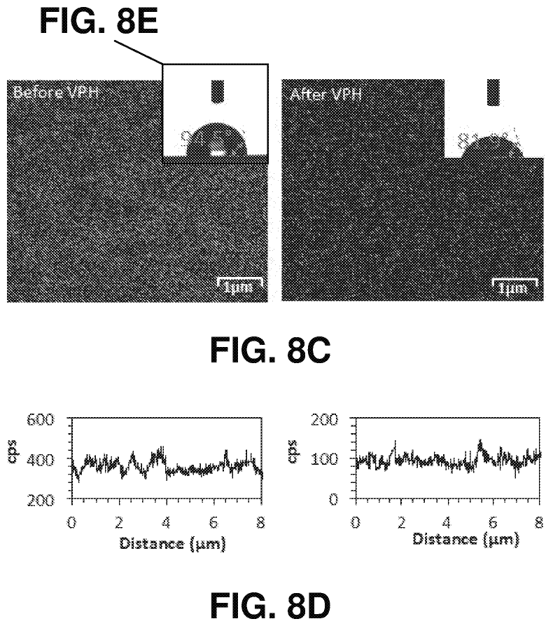

The Changes of the Membrane Properties Before and After Vapor-Phase Hydrolysis. As illustrated in FIG. 5, the membrane preparation protocol involved three consecutive steps: the modification of cellulose into TMSC to create a well-miscible polymer blend for solution coating, followed by PDMS curing, and eventually an acid-vapor hydrolysis (VPH) to transform the TMSC back to cellulose. As a polymer with Si—O bonds, TMSC bears similar chemical properties to PDMS, enabling the preparation of a highly compatible polymer mixture in the commonly used solvents like n-hexane. PDMS formation was performed through the catalytic hydrosilylation between the vinyl-terminated dimethylsiloxane and the hydrosilane-containing crosslinker (see FIG. 5). Curing at about 100° C. for about 2 hours was performed to complete the reaction. TMSC in the blend membrane was then regenerated back to cellulose through a vapor-phase hydrolysis (VPH) using hydrochloric acid. This step was necessary for TMSC to regain the cellulose structure, yet it could be detrimental for PDMS as it can lead to depolymerization. However, because of the higher reactivity, the acid vapor attacked preferentially the silyl group in TMSC rather than the siloxane positioned in the PDMS backbone. About a 15 min vapor hydrolysis was required to completely remove the silyl groups and restore the hydroxyl groups previously present in cellulose. To elucidate the TMSC transformation in the blend membrane, FTIR spectra of the 50RC membrane were recorded before and after vapor hydrolysis. From FIG. 8A, it was apparent that, upon hydrolysis, a strong O-H peak appeared between 3,000-3,700 cm⁻¹, which was accompanied by a strong decrease of the TMS-related peaks around 1,250 and 840 cm⁻¹, indicating that a significant amount of TMS was cleaved. These results were concomitant with the atomic composition obtained from XPS measurement (FIG. 8B), where the silicon percentage decreased by more than half upon cellulose regeneration.

In addition to the excellent compatibility, the blend membranes also demonstrated a good homogeneity, as seen in the large-scale silicon map recorded using EDX spectroscopy (FIG. 8C). The silicone-related compounds were represented as red dots. EDX line scan image presented in FIG. 8D shows that the silicone content of the membrane before VPH was significantly higher than after the reaction, supporting the aforementioned ATR-IR and XPS results. These chemistry changes were also followed by a decrease in the surface water contact angle (FIG. 8E), realized from the substitution of the hydrophobic siloxane groups by the hydrophilic hydroxyls.

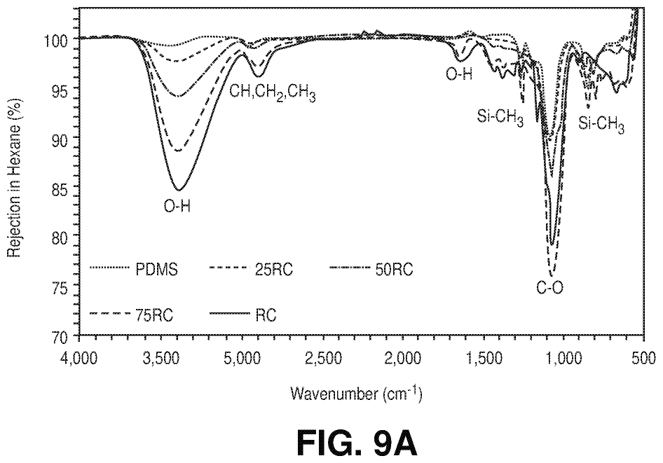

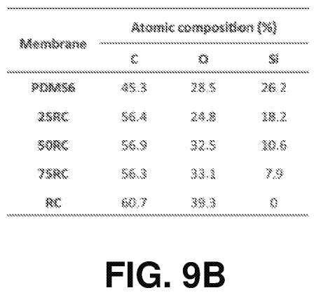

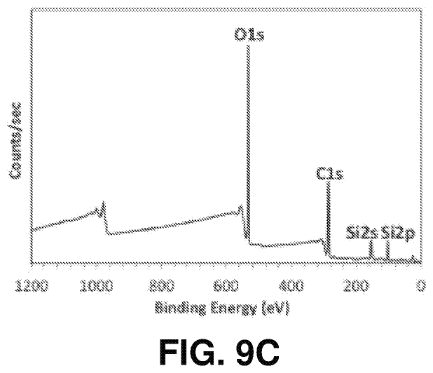

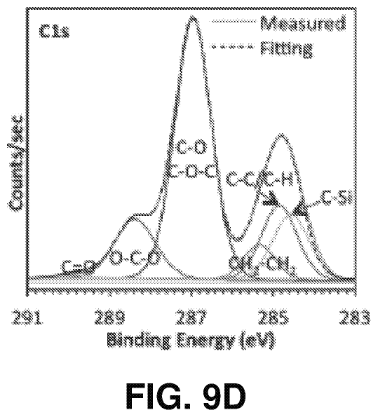

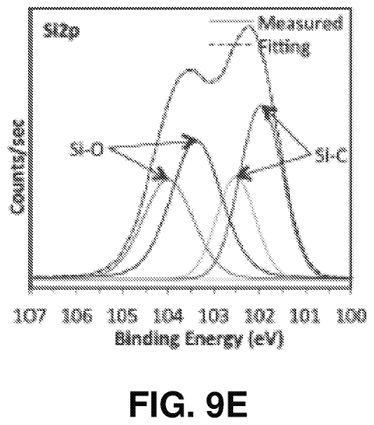

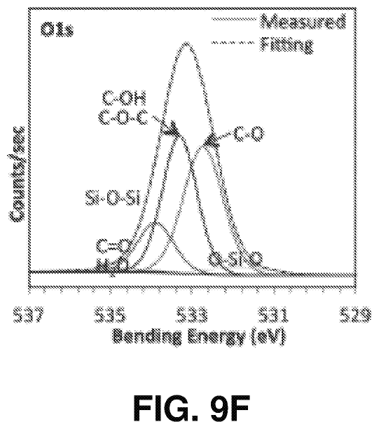

Final Membrane Structure and Morphology. Different compositions of TMSC and PDMS in the blend membranes were prepared and their final structure and morphology were characterized. The ATR-IR spectra of the pure and blend membranes are presented in FIG. 9A. Among the most intense peaks associated with PDMS were Si—CH₃ bands at about 1,260 and 795 cm⁻¹ as well as the asymmetric Si—O—Si stretching evidenced between 1,055-1,090 cm⁻¹. For the polymer blends, the spectra show the signature of cellulose bands between 3,050-3,700 cm⁻¹ and 1,060 cm⁻¹ attributed to the strong O—H and C—O bonds, respectively. With the increasing cellulose content in the membranes, the intensity of the cellulose characteristics band was higher accompanied by the simultaneous decrease in the intensity of the silicon-related peaks. This was also followed by the slightly higher intensity of the alkane related peaks around 2,900 cm⁻¹ attributed to CH, CH₂ and CH₃ as well as O—H bending vibration near 1,660 cm⁻¹ raised from the cellulose backbone. Further elucidation of the membrane chemistry was carried out by measuring the atomic composition of all membranes using XPS. The results presented in FIG. 9B clearly demonstrate the proportional decrease of silicon content in the membranes corresponding to their compositions. It can also be seen that the pure RC membrane did not contain any silicon, suggesting that the performed hydrolysis method allowed the complete transformation of TMSC back to cellulose. Accordingly, the silicon-related peaks appearing in the blend membranes were associated only with PDMS. Bonding configuration was performed on the 50RC membrane. FIG. 9C displays the XPS survey spectra, while FIGS. 9D-9F present the high-resolution curve fitting in the C1s, Si2p and O1s region respectively using the Lorentzian-Gaussian function. The high-resolution spectra revealed the presence of PDMS characteristic bonds consisting of C—Si and CH2—CH2 peak at 284.6 and 285.4 eV respectively and all the silicon related peaks in Si2p and O1s region. The intense peaks specifically attributed to cellulose consisted of the C—O related peaks at around 287 and 288.5 eV located in the C1s region, as well as near 534 and 533 eV in the O1s region.

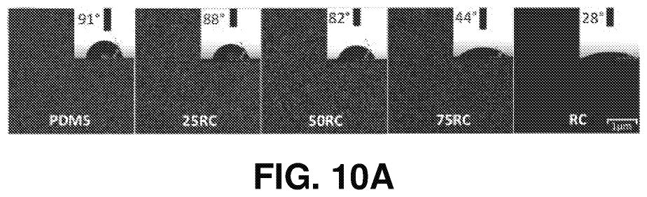

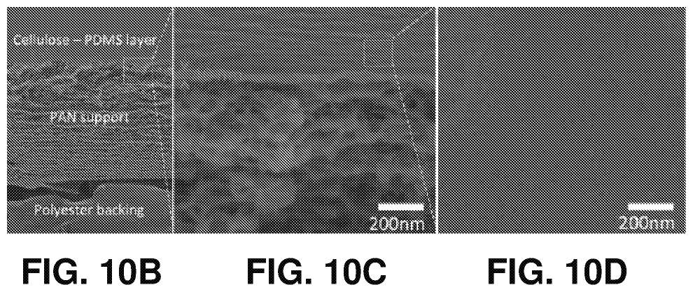

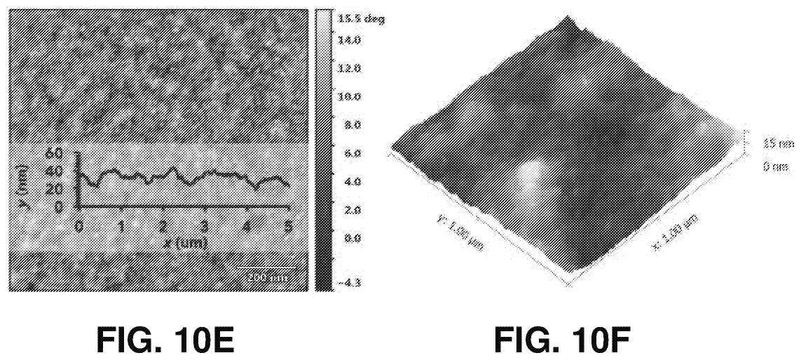

The introduction of cellulose in the membranes also significantly affected the final surface properties. It is well-accepted that membranes with a strong hydrophilic character tend to permeate polar solvents better than those with low polarity and vice versa. The membrane wettability was also responsible for the swelling phenomenon, which could severely affect the membrane performance. The data presented in FIGS. 10A-10F shows the decreasing water contact angle from around 80° for the pure PDMS, which was similar to previously reported values, to below 30° for the pure cellulose (RC). When cellulose concentrations increased in the 25RC and 50RC membranes, the surfaces were still very hydrophobic with the water contact angle slightly below that of PDMS. A significantly lower contact angles of about 40° was observed for the 75RC membrane. According to FIGS. 10B-10D, the 50RC membrane displayed a highly smooth layer without any observable defects. The active layer with an approximate thickness of 40 nm adhered very well to the porous support. The membrane also demonstrated a good uniformity as depicted by the AFM images (FIGS. 10E-10F).

OSN Performance. Organic solvent nanofiltration (OSN) is a relatively new yet promising membrane technology for the separation of molecules in the range of 200-1,000 Da. Recent advances in OSN have led to various applications in the pharmaceutical, petrochemical, biotechnology, food, and semi-conductor industry. The state-of-the-art OSN membranes are predominantly derived from polyimide in an integrally skinned asymmetric configuration. In these membranes, the selective and support layer are formed simultaneously from the same material, creating limitation in the solvent fluxes. Alternatively, OSN membranes can be fabricated as a thin film composite, allowing the optimization of the active layer independently from the support. Chemically stable membranes with high flux and rejection are necessary for a successful industrial application. Additionally, given that the typical OSN membranes can only be used for solvents with similar polarity, membranes that allow fast permeation of a wide range of solvents is highly desirable.

In this Example, membranes consisting of different ratios of cellulose and PDMS were evaluated in the filtration of different organic solvents. As listed in Table 1, the total polymer concentration in the coating solution was fixed to be 2 wt %, except for the case of pure PDMS membranes where a minimum solution concentration of 6 wt % was required to acquire defect-free membranes. The quality of the PDMS membranes was first confirmed by studying the oxygen and nitrogen permeation. The intrinsic O₂/N₂ selectivity of pure PDMS membranes, which is around 2.1, was achieved using 6 wt % solution concentration (PDMS6), whereas the selectivities of the PDMS2 and PDMS4 membranes were lower, indicating the presence of defects. Benefiting from the cellulose/TMSC properties, the cellulose-PDMS blend membranes can be fabricated with only 2 wt % solution concentration leading to thin membranes with high solvent fluxes.

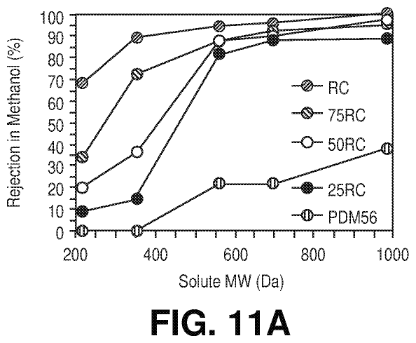

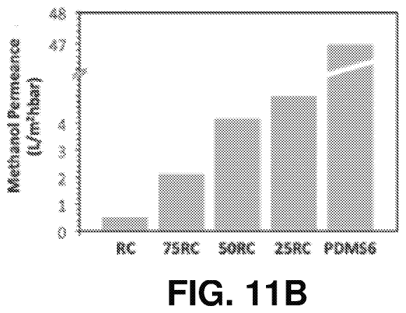

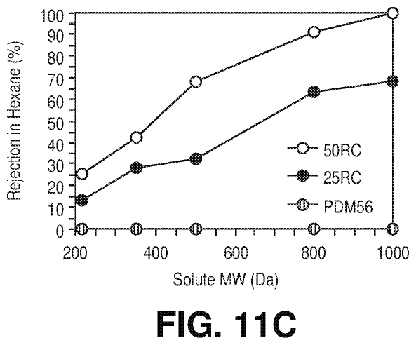

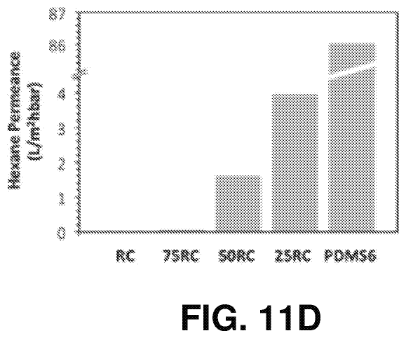

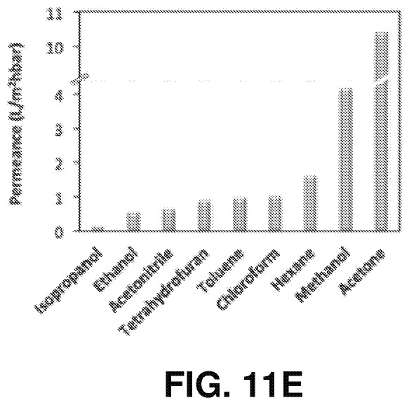

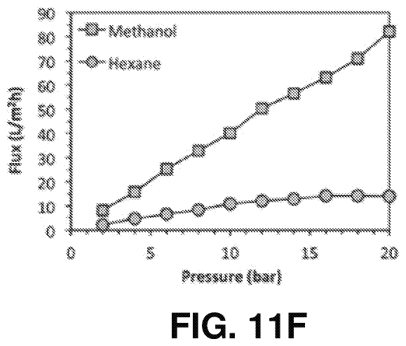

FIGS. 11A-11F reveal that the pure regenerated cellulose and the pure silicone rubber membrane show very different filtration performance as expected. The RC membrane exhibited a size selectivity with a molecular weight cut-off (MWCO) of around 300 Da combined with a methanol permeance of about 0.5 L/ m²hbar (FIGS. 11A-11B). In contrast, the PDMS6 membrane showed a very poor selectivity combined with a very high permeance. In the permeation of hexane (FIGS. 11C-11D), the RC membrane did not display any permeance as expected from its strong hydrophilic character (see FIG. 10A), while the PDMS6 membrane completely lost its selectivity due to excessive swelling.

Interestingly, a favorable combination between cellulose selectivity and PDMS flexibility was achieved using the blend membranes. The introduction of a small portion of TMSC in the 25RC membrane substantially improved the membrane sieving properties both in methanol and hexane filtration. By using neutral dyes in methanol, the membrane demonstrated more than 80% rejection of bromopryrogallol red (615 Da) and around 90% of tetrabromophenol blue (986 Da), suggesting an approximately 1,000 Da MWCO. Similar rejections were also obtained using the different neutral markers in hexane. Permeances as high as 4 and 5 L/m²hbar of pure methanol and pure hexane respectively were measured using this membrane. Increasing the cellulose percentage to 50% in the 50RC membrane further enhanced the rejection especially toward the solutes with molecular weight of 558 Da and higher, resulting in an estimated MWCO of about 750 Da in both solvents. The methanol permeance of this membrane was about 4.2 L/m²hbar for pure methanol and 1.6 L/m²hbar for pure hexane. With the domination of cellulose in the 75RC membrane, the MWCO was estimated to be about 650 Da in methanol with around 2 L/m²hbar permeance. However, the hexane permeance dropped significantly attributed to the dominant contribution of cellulose in the membrane wettability as indicated by its low water contact angle (FIG. 10A). On the one hand, the incorporation of cellulose into the PDMS matrix reduced the PDMS chain flexibility resulting in a better molecular sieving.

On the other hand, the presence of PDMS in the cellulose network interrupted the densely packed cellulose structure, promoting higher solvent fluxes especially in the case of nonpolar solvents (e.g. hexane). The 50/50 blend (50RC membrane) was a favorable trade-off showing good fluxes and selectivities in polar and non-polar solvents.

Encouraged by the good filtration results with methanol and hexane the 50RC membrane was tested with seven more solvents. Methanol and hexane, which represent the most polar and non-polar solvent among the list, gave reasonably high permeances, signifying that the membrane performances were barely controlled by the solvent polarity. This was because the membrane contained both highly polar and highly non-polar segments, allowing the easy passage of the solvent with any polarity. According to FIG. 11E, the highest permeance of 10.5 L/m²hbar was achieved using acetone, the lowest permeance of 0.1 L/m²hbar was obtained using isopropanol, which is the most viscous solvent tested in the study, while the rest of the solvents showed acceptable permeance values between 0.5-1 L/m²hbar. It is well known, that increasing solvent viscosity reduces the solvent permeance. However, acetone had the highest permeance in these experiments, but it did not have the lowest viscosity, indicating that other parameters such as solubility and solvent sizes affected the permeation rate. Considering that the rejection properties are similar for the filtration of methanol and hexane (FIGS. 11A, 11C), similar rejections were anticipated using other solvents, including acetone. Compared to the most studied commercial OSN membrane (MPF-50, MWCO 700, Koch, USA), the methanol flux of the 50RC membrane was more than four times higher, while the MWCO and acetone flux were similar. Moreover, unlike our membranes, MPF-50 was found to be completely incompatible with hexane.

The effect of the feed pressure on the flux of methanol and hexane through the 50RC membrane was tested between 2 and 20 bar (FIG. 11F). No compaction effect could be observed in the case of methanol filtration in the applied pressure range; a slight flux decline took place in the case of hexane at a pressure above 12 bar. Compaction is usually caused by the support layer; the small effects observed in this study can be attributed to the good structural feature of the PAN layer with the absence of macrovoids (see FIG. 10B-10C). Overall, the good OSN performance obtained by the blend membranes was based on the successful combination of the cellulose selectivity and PDMS permeability. The simple membrane fabrication and the fact that the membranes can be applied for polar and non-polar solvents broadens the range of OSN applications.

Pervaporation Performance. Motivated by the intriguing performance of the PDMS/cellulose blend membranes for organic solvent nanofiltration these membranes were tested for the separation of water-rich ethanol-water streams by pervaporation. In pervaporation the liquid feed stream contacts the membrane and the permeate is transported through the membrane, withdrawn as vapor and then condensed.

Industrially, classic distillation separates water-rich ethanol-water streams, and pressure-swing/azeotropic-extractive units are applied for ethanol-rich azeotropic mixtures (requiring massive use of energy/solvents). In pervaporation, a membrane enhances the evaporation of a target mixture-compound and ethanol can be separated from water consuming a fraction of the energy required for distillation; ideally, for a membrane with infinite selectivity, the energy could be limited to the heat of evaporation of the ethanol (or water) of the feed. Hydrophobic pervaporation of water-rich streams may be a breakthrough technology for bioethanol upgrading; this separation is difficult since water has a diffusion advantage, limiting ethanol enrichment. Many studies were already reported in the literature about ethanol selective membranes for pervaporation; mainly, PDMS-based membranes were studied in the supported/unsupported, blended, or in a mixed-matrix configuration.

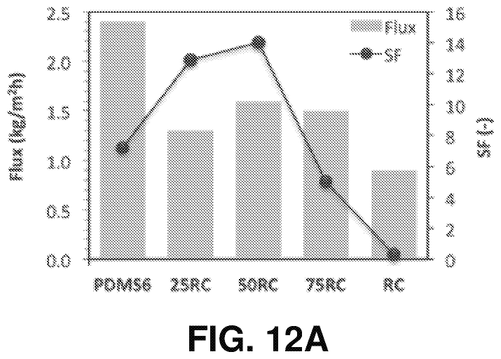

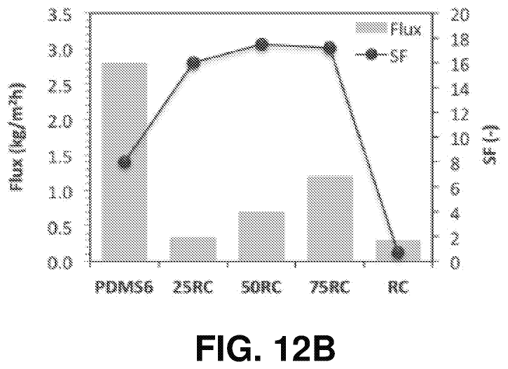

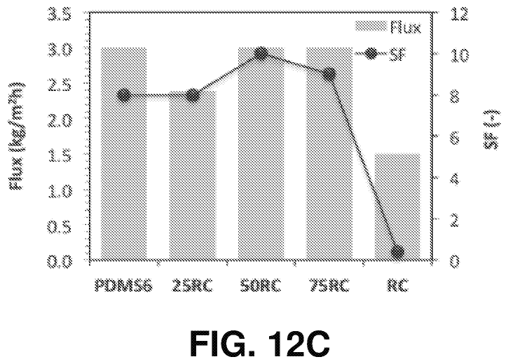

Two different feed mixtures containing 5 wt % and 10 wt % ethanol, which mimick the composition of the fermentation broths, were investigated at different operating temperatures. Different composition of cellulose-PDMS membranes were first evaluated in the pervaporation of aqueous feed containing 5 wt % ethanol at ambient temperature. Under these conditions the pure PDMS membrane (PDMS6) showed a separation factor of 7.2, similar to previously reported values, with a flux of 2.4 kg/m²h (FIG. 12A). On the other hand, the pure RC membrane expectedly gave a reverse (water selective) separation factor of 0.3 due to its strong hydrophilic property. Interestingly, blending PDMS with RC allows increasing the ability of the membrane to pervaporate ethanol. This result seemed to be counterintuitive since PDMS is hydrophobic while RC is hydrophilic, and it is speculated that the presence of the RC in the matrix forces water through slower diffusion paths (as schematically illustrated in FIGS. 4A-4B). In comparison with pure PDMS, water diffusivity was reduced and ethanol/water separation factor enhanced. With the presence of small percentage of cellulose in the 25RC membranes, the separation factor significantly increased to 12.9, accompanied by the flux decreased to 1.3 kg/m²h. A 50:50 ratio between the polymer components in the 50RC membrane led to even more interesting results where a separation factor of 14 was achieved, which doubled the separation factor of the pure PDMS, with just slightly compromising the PDMS flux to 1.6 kg/m²h. As a comparison, using a similar feed concentration at 30° C., the commercial Pervap 4060 (Sulzer ChemTech, Switzerland) and Pervatech PDMS (Pervatech BV, The Netherlands) had a flux of 0.6 and 1 kg/m²h with separation factors of 8.4 and 5.2, respectively.

FIGS. 12B-12C demonstrate the performance of the membranes using 10 wt % ethanol/water feed. With higher ethanol content in the feed running at 24° C., the performance of the pure membranes was similar to the one with 5% ethanol/water, while all the blend membranes presented remarkably higher separation factors with lower fluxes. The 50RC membrane exhibited the highest separation factor of 17.5, which was more than twice the separation factor of the pure PDMS membrane. Operating the experiment at a higher temperature of 60° C. expectedly led to higher fluxes and lower separation factors as presented in FIG. 12C. The higher fluxes in the experiments with higher temperature were attributed to the higher diffusion rate of the solvent molecules as well as the enhanced driving force across the membrane, whereas the separation factor decreased mainly because of the lower solubility selectivity. Remarkably, in this experiment the 50RC membrane displayed a similar flux as the PDMS6 membrane, while presenting a higher separation factor of 10. This emphasized that the introduction of cellulose into the PDMS matrix facilitated the selective ethanol transport while maintaining the exceptionally high flux of PDMS. More interestingly, the superiority of the blend membrane over the commercial membranes was again displayed using similar test conditions. The performance obtained with a 10% ethanol feed confirmed again that the PDMS/cellulose blend membrane was competitive with commercial membranes. Pervaporation of 10 wt % ethanol at 50° C. using the Pervap 4060 gave a 1.9 kg/m²h flux and 7 SF whereas the Pervatech PDMS showed a 3.3 kg/m²h flux with a lower SF of 6.

In conclusion, for the first time homogeneous blends of polydimethylsiloxane with cellulose are introduced. The blending of these very different polymers was possible via a “Trojan horse” method. The cellulose was first transformed into an amorphous hydrocarbon soluble polymer by functionalization with trimethylsilyl groups. The trimethylsilyl cellulose could then be blended in solution with PDMS. Composite membranes were manufactured by solution coating of a porous UF membrane. Finally the cellulose was regenerated by treatment with hydrochloric acid vapor leading to very thin unique PDMS/cellulose blend layers acting as selective membranes for organic solvent nanofiltration and pervaporation. The interplay between the two polymers—one hydrophilic and rigid, the other one hydrophobic and flexible—led to membranes with significantly enhanced separation performance when compared with the pure polymer membranes. In the case of organic solvent nanofiltration, the cellulose present in the interpenetrating PDMS/cellulose network reduced the swelling of the PDMS by the organic solvent and increased the membranes selectivity without large flux reduction. Due to the nature of the blend, the membranes can be used with polar and non-polar solvents. In the case of pervaporation of water/ethanol mixtures the addition of the water selective cellulose to PDMS surprisingly resulted in blend membranes with significantly enhanced ethanol separation ability. The presence of the cellulose in the matrix forced water through slower diffusion paths and in comparison with pure PDMS, water diffusivity was reduced and ethanol/water separation factor enhanced.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

1. A method of preparing a membrane, comprising:
contacting a cellulose precursor and a polydimethylsiloxane (PDMS) recursor in a solvent to form a polymer blend solution;
depositing the polymer blend solution on a surface of a suitable support;
curing the PDMS precursor of the polymer blend solution to form PDMS; and
converting the cellulose precursor to cellulose to form a polymer blend membrane including cellulose and PDMS.

2. The method of claim 1, wherein the cellulose precursor is a cellulose functionalized with trimethylsilyl (TMS), dimethylsilyl (DMS), triethylsilyl (TES), triisopropylsilyl (TIPS), t-butyldimethylsilyl (TBS/TBDMS), or t-butyldiphenylsilyl (TBDPS).

3. The method of claim 1, wherein the cellulose precursor is trimethylsilyl cellulose (TMSC), dimethylsilyl cellulose (DMSC), triethylsilyl cellulose (TESC), triisopropylsilyl cellulose (TIPSC), t-butyldimethylsilyl cellulose (TBDMSC), or t-butyldiphenylsilyl cellulose (TBDPSC).

4. The method of claim 1, wherein the PDMS precursor includes a pre-polymer and a crosslinker.

5. The method of claim 1, wherein the solvent is an organic solvent.

6. The method of claim 1, wherein the polymer blend solution is a compatible miscible polymer blend.

7. The method of claim 1, wherein the polymer blend solution exhibits no visible phase separation.

8. The method of claim 1, wherein the suitable support is a polymeric support.

9. The method of claim 1, wherein the PDMS is functionalized with end groups selected from the group consisting of vinyl, hydride (—H), methyl (—CH₃), 3-aminopropyl (—CH₂CH₂CH₂NH₂), glycidyl ether (—CH₂CH₂CH₂OCH₂), hydroxyl (—OH), alkyhydroxy (—(CH₂)ₘOH), chloride (—Cl), or stearate

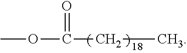

10. The method of claim 1, wherein the PDMS is a copolymer with functional groups selected from the group consisting of hydride (—H), 3-(2-(2-hydroxyethoxy)ethoxy)propyl (—CH₂CH₂CH₂(OCH₂CH₂)₂OH), poly(ethylene glycol) methyl ether (—CH₂CH₂CH₂O(CH₂CH₂O)ₙ CH₃), 3-aminopropyl, —CH₂CH₂CH₂NH₂, and polyacrylates.

11. The method of claim 1, wherein the polymer blend membrane includes regenerated cellulose.

12. The method of claim 1, wherein the polymer blend membrane comprises a layer including a polymer blend of cellulose and PDMS and a support in contact with the layer.

13. The method of claim 1, wherein the converting is achieved by one or more of vapor-phase hydrolysis, reintroducing active hydrogen, immersion in 1M hydrochloric acid solution, and hydrolysis with boiling water and simple alcohols.

14. A method of separating chemical species by organic solvent nanofiltration, comprising:
contacting a first side of a polymer blend membrane with a feed stream containing one or more solutes dissolved in an organic solvent; and
collecting a permeate from a second side of the membrane;
wherein the polymer blend membrane comprises a layer including a polymer blend of regenerated cellulose and PDMS and a support in contact with the layer.

15. The method of claim 14, wherein the first side of the polymer blend membrane includes the layer including the polymer blend of regenerated cellulose and PDMS.

16. The method of claim 14, wherein the organic solvent includes one or more of isopropanol, ethanol, acetonitrile, tetrahydrofuran, toluene, chloroform, hexane, methanol, and acetone.

17. The method of claim 14, wherein a concentration of the regenerated cellulose ranges from about 25 wt % to about 75 wt % of the total polymer concentration of the polymer blend membrane.

18. A method of separating chemical species by pervaporation, comprising:
contacting a first side of a polymer blend membrane with a liquid feed stream containing one or more solvents; and
collecting a permeate in a vapor or gas phase from a second side of the membrane;
wherein the polymer blend membrane comprises a layer including a polymer blend of regenerated cellulose and PDMS and a support in contact with the layer.

19. The method claim 18, wherein the first side of the polymer blend membrane includes the layer including the polymer blend of regenerated cellulose and PDMS.

20. The method of claim 18, wherein the liquid feed stream includes water and ethanol.
